(19) United States
(12) Plant Patent Application Publication
(10) Pub. No.: US 2004/0237155 P1
(43) Pub. Date: Nov. 25, 2004

(54) Climber rose plant named 'HARyup'

(76) Inventors: Robert B. Harkness, Mougins (FR); Philip A. Harkness, Hitchin (GB)

Correspondence Address: KLARQUIST SPARKMAN, LLP, Suite 1600, One World Trade Center, 121 S.W. Salmon Street, Portland, OR, 97204, US

(21) Appl. No.: 10/648,412

(22) Filed: Aug. 25, 2003

Related U.S. Application Data
(63) Continuation of application No. 09/255,107, filed on Feb. 22, 1999.

(30) Foreign Application Priority Data
Jul. 20, 1992 (GB) 5/1357

(51) Int. Cl. A01H 5/00

(52) U.S. Cl. PLT/114

(57) ABSTRACT

Climber rose plant having good floriferousness, resistance to mildew and rust, vigorous growth habit suitable for a climbing rose, and a novel soft pink flower color.

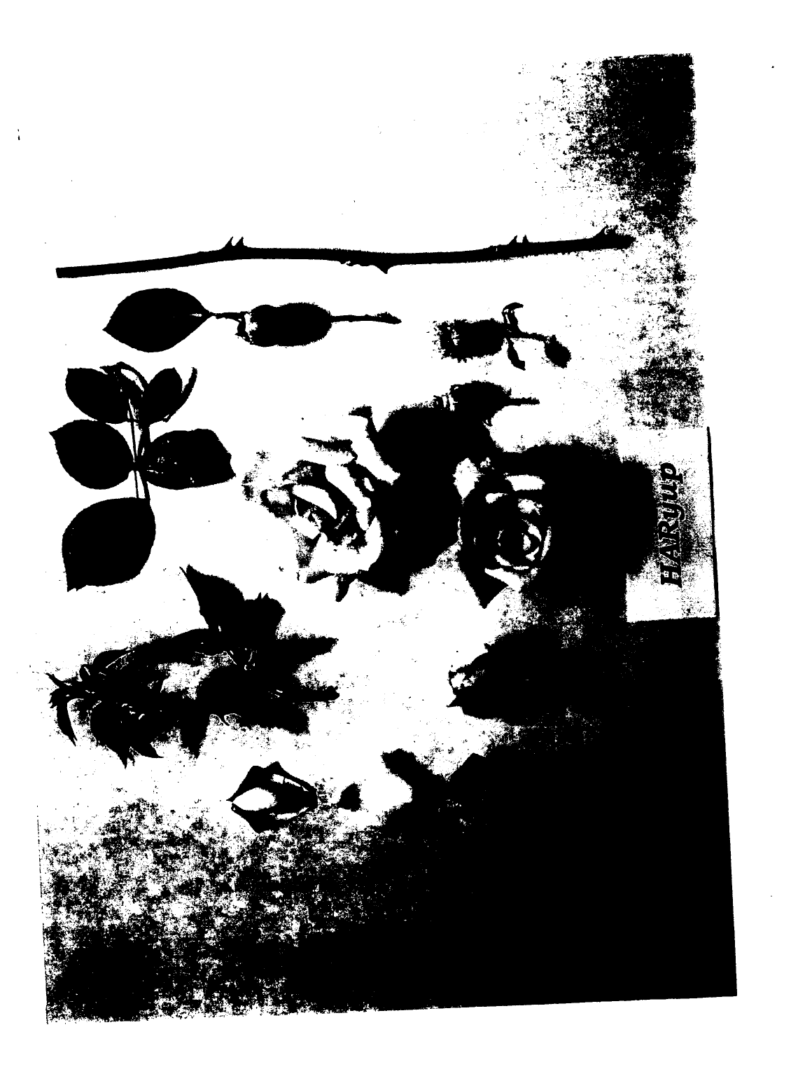

The present invention relates to a new and distinct variety of rose plant of the climber class which was originated by us by crossing the variety Compassion, not patented, with the variety Congratulations, not patented.

The primary objective of this breeding was to produce a new rose variety having improved floriferousness, improved resistance to disease, vigorous growth habit suitable for a climbing rose, and a novel soft pink flower color. The objective was substantially achieved, along with other desirable improvements, as evidenced by the following unique combination of characteristics that are outstanding in the new variety and that distinguish it from its parents, as well as from all other varieties of which we are aware:

1. Climbing habit—flexible, very long canes;

2. Hybrid tea flower form;

3. Soft, warm pink flower color;

4. Season-long bloom production (good repeat bloom); and

5. Dark green, glossy, disease resistant foliage.

‘HARyup’ forms a large climbing rose plant in U.S. Department of Agriculture Zones 5-10. Its long, climbing canes are suitable for training on a trellis. Lateral buds will break freely as the canes are trained along the trellis giving a tremendous show of bloom in the spring followed by good repeat bloom until frost stops growth.

Asexual reproduction of this new variety by budding, as performed at Wasco, Calif., shows that the foregoing and all other characteristics and distinctions come true to form and are established and transmitted through succeeding propagations.

The accompanying illustration shows typical specimens of the vegetative growth and flowers of this new variety in different stages of development, depicted in color as nearly true as it is reasonably possible to make the same in a color illustration of this character.

The following is a detailed description of our new rose cultivar with color descriptions using terminology in accordance with The Royal Horticultural Society (London) colour chart, except where ordinary dictionary significance of color is indicated.

Parentage:

Seed parent.—Compassion (patent not applied for).

Pollen parent.—Congratulations (patent not applied for).

Classification:

Botanical.—Rosa Hybrida.

Commercial.—Climber.

### FLOWER

Observations made from specimens grown in a garden environment at Somis, Calif. in January 1996-December 1998.

Blooming habit: Continuous.

Bud:

Size.—One and one-half inches long when the petals start to unfurl.

Form.—The bud form is long, pointed ovoid.

Color.—When sepals first divide, bud color is Red-Purple Group 58D. When half blown, the upper sides of the petals are, on the outermost row of petals, Red-Purple Group 58D. The upper sides of the inner petals are Red Group 49C. The lower sides of the petals are on the outermost row of petals, Red-Purple Group 58D. The lower sides of the inner petals are Red Group 49B.

Sepals.—Color: Green Group 138C except when exposed to bright sun. Then they have a bronze caste, close to Greyed-Purple Group 183C. Surface texture: Covered in many fine hairs. There are three lightly appendaged sepals. There are two unappendaged sepals with hairy edges.

Receptacle.—Color: Greyed-Purple Group 183C. Shape: Apple. Size: Medium, ⅜″×5/16″. Surface: Smooth.

Peduncle.—Length: Short, 1 to 1½ inches. Surface: Glandular. Color: Greyed-Purple Group 183C to 183D. Strength: Weak, bending.

Bloom:

Size.—Large. Average open size is four inches.

Borne.—Singly and several together in rounded clusters of three to five blooms.

Stems.—Strength: Strong. Average length is about twelve to fourteen inches when they come off of the long climbing canes which are four to seven feet in length.

Form.—When first open: High-centered. Permanence: Retains its form to the end, outer petals curl back.

Petalage.—Number of petals under normal conditions: 20.

Color.—The upper sides of the petals are, on the outermost row, Red-Purple Group 58D. The upper sides of the inner petals are Red Group 49C. The reverse sides of the petals are, on the outermost row, Red-Purple Group 58D. The reverse side of the inner petals are Red Group 49B. The base of each petal has on the upper side a large (¼″×⅜″) half moon, close to Yellow Group 6C. The lower side of each petal has a small (¼″×3/16″) half moon, close to Yellow Group 2C. The major color on the upper side is Red Group 49C.

Variegations.—Occasional spots on the petals where damage from wind or insects has occurred.

Discoloration.—The general tonality at the end of the first day: No change. At the end of the third day: Little if any change. By day 5, the color will fade to Red Group 49D except on the outermost petal row which fades to Red-Purple Group 62C.

Fragrance.—Moderate, spicy.

Petals:

Texture.—Thick.

Shape.—Round to oval.

Form.—Tips slightly recurved.

Arrangement.—Imbricated. Petaloids in the center: None.

Persistence.—Petals drop off cleanly before drying.

Lastingness.—On the plant: Fair (about five to six days). As a cut flower: Fair (about five to six days).

Reproductive parts:

Anthers.—Size: Large. Quantity: Many. Color: Orange-Yellow Group 20A. Arrangement: Regular around styles.

Filaments.—Color: Red Group 49D.

Pollen.—Color: Not tested.

Styles.—Color: Red Group 53B.

Stigmas.—Color: Yellow-White Group 158C.

Plant:

Form.—Climber.

Growth.—Vigorous. Height of mature plant is ten feet, width is twelve to fourteen feet, trained upright and arching on a trellis.

Foliage: Number of leaflets on normal mid-stem leaves: 5.

Size.—Large (5½″ long×4½″ wide).

Quantity.—Abundant.

Color.—New foliage: Upper and lower surfaces: Greyed-Purple Group 183B Old foliage: Upper surface: Green Group 137A. Lower surface: Yellow-Green Group 146B.

Leaflets:

Shape.—Pointed oval.

Texture.—Leathery, glossy.

Edge.—Serrated.

Serration.—Single, small.

Petiole rachis.—Color: Yellow-Green Group 146B.

Petiole underside.—Smooth.

Stipules.—Long, (about one inch), serrated.

Disease resistance.—Resistant to mildew and rust under normal growing conditions. Susceptible to blackspot.

Wood:

New wood.—Color: Greyed-Purple Group 183B. Bark: Smooth.

Old wood.—Color: Yellow-Green Group 146B, but will color in bright sunlight to Greyed-Red Group 178A. Bark: Smooth.

Prickles:

Quantity.—On main canes from base: Ordinary. On laterals from main canes: Ordinary.

Form.—Hooked downward.

Color when young.—Greyed-Purple Group 183B.

Color when mature.—Greyed-Orange Group 166A.

Small prickles:

Quantity.—On main stalks: None. On laterals: None.

1. A new and distinct variety of rose plant of the climber class, substantially as herein shown and described, characterized particularly by good floriferousness, resistance to mildew and rust, vigorous growth habit suitable for a climbing rose, and a novel soft pink flower color.
